(19) United States
(12) Patent Application Publication
(10) Pub. No.: US 2005/0130213 A1
(43) Pub. Date: Jun. 16, 2005

(54) Selective ligation and amplification assay

(76) Inventor: Tom Morrison, Winchester, MA (US)

Correspondence Address: BROMBERG & SUNSTEIN LLP, 125 SUMMER STREET, BOSTON, MA, 02110-1618, US

(21) Appl. No.: 11/010,201

(22) Filed: Dec. 10, 2004

Related U.S. Application Data
(60) Provisional application No. 60/528,461, filed on Dec. 10, 2003.

(51) Int. Cl. C12Q 1/68; C07H 21/04; C12P 19/34

(52) U.S. Cl. 435/6; 435/91.2; 536/25.32

(57) ABSTRACT

An improved assay for identifying and distinguishing one or more a single nucleotide polymorphisms in one or more target sequences of nucleic acid comprises, in a single-tube reaction system, three or more primers, two of which bind to a target nucleic acid sequence, flanking a SNP, so that the 3′-end of one or more first primers is adjacent to the 5′-end of a second primer, the two primers being selectively ligated and then amplified by a third primer to exponentially produce the complementary strand of the one or more target sequences. The other strand of the one or more target sequences are exponentially amplified by one or more hybridizable probes, each labeled with a different fluorophore, the fluorophore-labeled hybridizable probes being quenched until incorporation into and amplification of target nucleic acid products. Also provided is a method for identifying one or more SNPs in one or more target sequences of nucleic acid in each single through-hole of a nanoliter sampling array, and a kit for such a method containing a nanoliter sampling array chip, primer sequences, and reagents required to selectively ligate primers for amplification of desired target nucleic acid sequences.

### RELATED APPLICATIONS

The present application claims priority from provisional patent application Ser. No. 60/528,461, filed Dec. 10, 2003 and from provisional patent application Ser. No. 60/531,726, filed Dec. 22, 2003, both of which are hereby incorporated by reference herein in their entirety.

### TECHNICAL FIELD

The present invention relates to assays for amplifying and identifying target sequences of nucleic acids involving a combined ligation and amplification protocol, and the use of nanoliter sampling arrays to perform such assays.

### BACKGROUND ART

Genetic variations are increasingly being linked to a multitude of disease conditions and predispositions for disease, including cancer, multiple sclerosis, autoimmune diseases, cystic fibrosis, and schizophrenia. The ability to identify genetic variations rapidly and inexpensively will greatly facilitate diagnosis, risk assessment, and determination of the prognosis for such diseases and predispositions for these diseases.

One possibility for identifying genetic variations involves combining selective ligation and amplification techniques, disclosed in U.S. Pat. No. 5,593,840 to Bhatnagar et al. and U.S. Pat. No. 6,245,505 to Todd et al, both of which are hereby incorporated by reference herein. Both patents disclose the use of at least three primers, two of which are complementary to adjacent regions of the 3′-end of one strand of a target nucleic acid sequence which, after hybridization, can be ligated and then extended. In Todd et al., the third primer is a random sequence, complementary to the random sequence at the 3′-end of the downstream primer (that ligates to the upstream primer) and identical to the random sequence on the 5′-end of the first primer. In Bhatnagar et al., the third primer is complementary to the upstream primer, and also to the opposite strand of the target sequence. In both cases, there must be complementarity at the 3′-end of the third primer to allow amplification to occur.

A heat-stable polymerase is used to amplify the target nucleic acid sequence, and both the ligation and amplification reactions can be carried out in the same reaction mixture. An optional gap between the adjacent primers may be present, which may be filled by a polymerase to allow successful ligation of the adjacent primers. Such a system allows identification of genetic variability in target nucleic acid sequences, and identification of multiple alleles.

### SUMMARY OF THE INVENTION

In a first embodiment of the invention, there is provided an improved assay of the type for amplifying a specific target nucleic acid sequence, wherein the target sequence comprises an internal SNP of interest, the assay being a selective ligation and amplification method of the type using a controlled-temperature reaction mixture including the target sequence, ligatable first and second primers having at least a portion substantially complementary to first and second segments of the target sequence, respectively, and a third primer that is substantially complementary to a random sequence segment of the first and second primers, wherein the improvement comprises: distinguishing in a single-tube reaction system between one or more SNPs in one or more target sequences of nucleic acid using two unique probes designed to hybridize to the target nucleic acid sequences with SNPs of interest, each hybridizable probe having a different fluorescent tag that is quenched until incorporation of the probe into amplified target nucleic acid product.

In some embodiments of the improved assay, of the type for amplifying a specific target nucleic acid sequence, wherein the target sequence comprises one or more SNPs of interest that are not at an end of the target sequence, the assay being a selective ligation and amplification method of the type using a thermocycled reaction mixture including the target sequence, a first primer having at least a portion of its 3′-end substantially complementary to a first segment at a first end of the target sequence, a second primer having at least a portion of its 5′-end substantially complementary to a second segment at a second end of the target sequence, the 5′-end of the second primer being adjacent to or within two to four bases of the 3′-end of the first primer wherein a nucleotide complementary to the SNP of the target sequence is present at either the 3′-end of the first primer or at the 5′-end of the second primer, and a third primer that is substantially complementary to a random sequence segment at the 3′-end of the second primer and to a substantially similar sequence at the 5′-end of the first primer, at least four different nucleotide bases, a thermostable polymerase and a thermostable ligase, wherein the improvement comprises distinguishing in a single-tube reaction system between one or more SNPs in one or more target sequences of nucleic acid using two unique probes designed to hybridize to the target nucleic acid sequences with SNPs of interest, each hybridizable probe having a different fluorescent tag that is quenched until incorporation of the probe into amplified target nucleic acid product. The first hybridizable probe with first fluorescent tag has a unique random sequence that hybridizes to a first amplified target nucleic acid generated by the third primer from a ligated first primer-second primer product having a first SNP of interest on the 3′-end of the first primer, the first hybridizable probe thereby becoming incorporated into amplified opposite strand target nucleic acid product to give a first fluorescent signal. The second hybridizable probe with second fluorescent tag has a unique random sequence that hybridizes to a second amplified product generated by the third primer from a different ligated first primer-second primer product having a second SNP of interest on the 3′-end of the first primer, the second hybridizable probe thereby becoming incorporated into amplified opposite strand target nucleic acid product to give a second fluorescent signal.

In a preferred embodiment, the random sequences of the first and second hybridizable probes are unique sequences, such that specific incorporation of each of the hybridizable probes into amplified target nucleic acid preferentially occurs after ligation of the first primer-second primer product having the particular SNP of interest that the hybridizable probe was designed to detect. Upon incorporation of the hybridizable probe into amplified product, fluorescence occurs, making detection of the amplified product distinguishable from non-specific background products. Additionally, the random sequence of the third primer is also a unique sequence, optimized for PCR to reduce non-specific amplified products that may be generated in the presence of human or other species chromosomes to a sufficiently low level that such non-specific products do not interfere with detection of amplified products having a SNP of interest.

Alternatively, the two hybridizable probes do not contain fluorescent tags, but are simply additional primers designed to distinguish different ligated products having different SNPs of interest. Detection of amplified product with a SNP of interest is then done using additional hybridizable probes, similar to the additional primers, but are developed in a manner not to interfere with amplification. These hybridizable probes have a fluorescent tag, or alternatively, each have a different fluorescent tag, and upon hybridizing to amplified product, fluoresce, thereby allowing detection of amplified product.

In another embodiment of the invention there is provided an improved assay of the type for amplifying a specific target nucleic acid sequence, wherein the target sequence comprises a SNP of interest that is not at an end of the target sequence, the assay being a selective ligation and amplification method of the type using a thermocycled reaction mixture including the target sequence, a first primer having at least a portion of its 3′-end substantially complementary to a first segment at a first end of the target sequence, a second primer having at least a portion of its 5′-end substantially complementary to a second segment at a second end of the target sequence, the 5′-end of the second primer being adjacent to the 3′-end of the first primer wherein a nucleotide complementary to the SNP of the target sequence is present at either the 3′-end of the first primer or at the 5′-end of the second primer, and a third primer that is substantially complementary to a random sequence segment at the 3′-end of the second primer and to a substantially similar sequence at the 5′-end of the first primer, at least four different nucleotide bases, a thermostable polymerase and a thermostable ligase, wherein the improvement comprises homogeneously detecting amplified target sequence using a dye specific for binding to double-stranded (ds) DNA that fluoresces upon binding target sequence. In a preferred embodiment, the random sequence of the third primer is a unique sequence, optimized for PCR such that no non-specific products are generated in the presence of human or other species chromosomes. In some embodiments, primers may be affixed on, within or under a biocompatible material such as a wax-like coating on the surface of the through-holes by drying the primers after application to the through-holes, wherein the biocompatible material may comprise, for example, a polyethylene glycol (PEG) material.

Alternatively, assays in accordance with the present invention may use a thermostable polymerase that lacks 5′ to 3′ exonuclease activity, or a thermostable polymerase that lacks 3′ to 5′ exonuclease activity, or a thermostable polymerase that lacks both 5′ to 3′ and 3′ to 5′ exonuclease activity. Examples of thermostable polymerases which lack 5′ to 3′ exonuclease activity include Stoffel fragment, Isis™ DNA polymerase, Pyra™ exo(−) DNA polymerase, and Q-BioTaq™ DNA polymerase. Examples of thermostable polymerases which lack 3′ to 5′ exonuclease activity include Taq polymerase, SurePrime™ Polymerase, and Q-BioTaq™ DNA polymerase. An example of a thermostable polymerase which lacks both 5′ to 3′ and 3′ to 5′ exonuclease activity is Q-BioTaq™ DNA polymerase. Suitable dyes include SYBR® Green I and SYBR® Green II, YOYO®-1, TOTO®-1, POPO®-3, ethidium bromide, or any other dye that allows rapid, sensitive detection of amplified target nucleic acid sequence using fluorescence.

In another embodiment, there is provided a nanoliter sampling array comprising a first platen having at least one hydrophobic surface and having a high-density microfluidic array of hydrophilic through-holes. In this particular embodiment, each through-hole contains at least a first primer having at least a portion of its 3′-end substantially complementary to a first segment at a first end of a potential nucleic acid target sequence a second primer having at least a portion of its 5′-end substantially complementary to a second segment at a second end of the potential nucleic acid target sequence, the 5′-end of the second primer being adjacent to the 3′-end of the first primer upon binding to the potential nucleic acid target sequence.

In addition, the sampling array may further comprise a second platen having at least one hydrophobic surface and having a high-density microfluidic array of hydrophilic through-holes wherein the first and second platen are fixedly coupled such that the through-holes of each are aligned.

In yet another embodiment, there is provided a method of identifying a SNP in a target sequence of nucleic acid, the method comprising providing a first sample platen having a high-density microfluidic array of through-holes, each through-hole having a first primer having at least a portion of its 3′-end substantially complementary to a first segment at a first end of the target sequence, a second primer having at least a portion of its 5′-end substantially complementary to a second segment at a second end of the target sequence, the 5′-end of the second primer being adjacent to the 3′-end of the first primer, and third primer that is substantially complementary to a random sequence segment at the 3′-end of the second primer and to the 5′-end of the first primer, introducing a sample containing a target sequence of nucleic acid having a SNP of interest to the array, introducing reagents to the through-holes in the array, the reagents including a thermostable polymerase, a thermostable ligase, and at least four different nucleotide bases, thermocycling the array, and detecting amplified target sequence. In a preferred embodiment, primers 1 and 2 are designed with a possible match to the target strand SNP located at either the 3′-end of the 5′ primer (the first primer) or located at the 5′-end of the 3′ primer (the second primer). When the first and second primers hybridize to the target strand, adjacent to each other and flanking the SNP, ligation of the primers only occurs if there is a successful match to the SNP by one of the primers. In this way, the ligation is selective and so selective amplification of the desired target sequence containing the SNP of interest also occurs. As described above, in some embodiments, primers may be affixed on, within or under a biocompatible material such as a wax-like coating on the surface of the through-holes by drying the primers after application to the through-holes, wherein the biocompatible material may comprise, for example, a polyethylene glycol (PEG) material.

In addition, the method of identifying a SNP in a target sequence of nucleic acid may additionally comprise using a thermostable polymerase that lacks 5′ to 3′ exonuclease activity, and detecting amplified target sequence using a dye specific for binding to double-stranded (ds) DNA that fluoresces upon binding target sequence. Alternatively, detecting may comprise using first primers and second primers designed to generate amplified target sequences with differential melting curves to distinguish individual amplified target sequences by differences in melting temperatures (Tₘs), or may comprise using a probe specific for hybridizing across a ligation junction formed between the first primer and second primer after binding to the target sequence wherein the probe specific for hybridizing across the ligation junction has a fluorescent group and a fluorescence-modifying group, or using a probe containing a fluorescent group and a fluorescence-modifying group specific for hybridizing to a region of the target sequence wherein upon extension of the probe, the fluorescence-modifying group is excised and the fluorescent group fluoresces. Additionally, detection may be done using a probe specific for hybridizing to any unique sequence in the amplified target nucleic acid, the probe having a fluorescent group and a fluorescence-modifying group such that the upon hybridization the probe fluoresces, allowing detection of the amplified target nucleic acid.

Other means of detection comprise the use of amplification primers which match the random sequence of primer 2 wherein the primers are labeled with a fluorescent group that only fluoresces when incorporated in a PCR product, similar to Lux™ primers known in the art. In such an embodiment, the fluorescent group is quenched by secondary structure before incorporation into double-stranded product, such that prior to incorporation, a sequence in the primer/probe binds to a complementary sequence in the primer/probe containing the fluorescent group, quenching the fluorescent group. In another embodiment, primers 1 and 2 are Fluorescence Resonance Energy Transfter (FRET) partners, such that when hybridized to the amplified target sequence, produced only after primers 1 and 2 are ligated and amplified, they fluoresce.

Yet another embodiment provides a kit for use in identification of amplified target nucleic acid sequences, the kit comprising a sample platen having one hydrophobic surface and having a high-density microfluidic array of hydrophilic through-holes. In the array of the kit, each through-hole contains at least a first primer having at least a portion of its 3′-end substantially complementary to a first segment at a first end of potential nucleic acid target sequence, and a second primer having at least a portion of its 5′-end substantially complementary to a second segment at a second end of the potential nucleic acid target sequence, the 5′-end of the second primer being adjacent to the 3′-end of the first primer upon binding to the potential nucleic acid target sequence. The kit also comprises a reagent platen having a high-density microfluidic array of through-holes, each through-hole containing a third primer that is substantially complementary to a random sequence segment at the 3′-end of the second primer and to a substantially similar sequence at the 5′-end of the first primer, at least four different nucleotide bases, a thermostable polymerase, and a thermostable ligase. In the kit of this embodiment, the reagent platen has a structural geometry that corresponds to the sample platen, thereby allowing delivery of reagent components and target nucleic acid sample to the primers in the sample platen. In other embodiments, the thermostable polymerase may lack 5′ to 3′ exonuclease activity.

### BRIEF DESCRIPTION OF THE DRAWINGS

The foregoing features of the invention will be more readily understood by reference to the following detailed description, taken with reference to the accompanying drawings, in which:

FIG. 1-A, shows a double-stranded target nucleic acid sequence with a single nucleotide polymorphism (SNP).

FIG. 1-B1 shows a denatured 3′ to 5′ target strand with primers 1 and 2 hybridized adjacent to the SNP, the base complementary to the SNP located at the 3′-end of primer 1 and shows the random sequence (RS) of primer 3 hybridized to 3′-end of the ligated P1-P2 product.

FIG. 1-B2 shows a denatured 3′ to 5′ target strand with primers 1 and 2 hybridized adjacent to the SNP, the base complementary to the SNP located at the 5′-end of primer 2 and shows the random sequence (RS) of primer 3 hybridized to 3′-end of the ligated P1-P2 product.

FIG. 1-C shows a denatured 5′-3′ target nucleic acid strand being extended by un-ligated primer P1.

FIG. 2-A shows a double-stranded target nucleic acid sequence with a single nucleotide polymorphism (SNP).

FIG. 2-B shows primers P1 and P2 hybridized to a denatured target strand of nucleic acid (the 3′ to 5′ strand) wherein a base complementary to the SNP in the target strand is present on the 3′-end of P1, and each of primers P1 and P2 contain a random sequence at their 5′-end and 3′-end, respectively.

FIG. 2-C shows ligated P1-P2 product being amplified by primer P3 to produce P3-amplified product.

FIG. 2-D shows P3-amplified product being amplified by primer P3 to produce P3-ampflied product (3′ to 5′).

FIGS. 2-E1 and 2-E2 show exponential amplification of P3-amplified product (5′ to 3′) and P3-amplified product (3′ to 5′), respectively.

FIG. 3 shows a cartoon of the dye SYBR® Green I binding to double-stranded amplified target nucleic acid and fluorescing.

FIG. 4-A shows upstream primer A-B, downstream primer C-D, and general extension primer D′ with a target nucleic acid having a SNP of interest in a single-tube reaction system for distinguishing between one or more SNPs in one or more target sequences of nucleic acid, the single-tube reaction system also containing upstream primer F-E and a second nucleic acid target with a second SNP of interest.

FIG. 4-B shows ligation of upstream primer A-B with downstream primer C-D when successful match-up occurs with a first SNP of interest in a first target sequence of nucleic acid, and also shows ligation of upstream primer F-E with down stream primer C-D when successful match-up occurs with a second SNP of interest in a second target sequence of nucleic acid present in the same tube.

FIG. 4-C shows extension of ligation products A-B-C-D and F-E-C-D by general extension primer D′.

FIG. 4-D shows hybridization of hybridizable probe A with fluorescent tag 1 to extended product A′-B′-C′-D′ and hybridization of hybridizable probe F with fluorescent tag 2 to extended product F′-E′-C′-D′.

FIG. 4-E shows incorporation and amplification of a first target nucleic acid with a first SNP of interest by hybridizable probe A, triggering fluorescence of fluorophore 1 in a first amplified product, and incorporation and amplification of a second target nucleic acid with a second SNP of interest by hybridizable probe F, triggering fluorescence of fluorophore 2 in a second amplified product.

FIG. 5A shows upstream primer A-B, downstream primer C-D, and general extension primer D′ with a target nucleic acid having a SNP of interest in a single-tube reaction system for distinguishing between one or more SNPs in one or more target sequences of nucleic acid the single-tube reaction system also containing upstream primer F-E and a second nucleic acid target with a second SNP of interest in an alternative embodiment of the single-tube reaction system of FIG. 4.

FIG. 5B shows ligation of upstream primer A-B with downstream primer C-D when successful match-up occurs with a first SNP of interest in a first target sequence of nucleic acid, and also shows ligation of upstream primer F-E with down stream primer C-D when successful match-up occurs with a second SNP of interest in a second target sequence of nucleic acid present in the same tube.

FIG. 5C shows extension of ligation products A-B-C-D and F-E-C-D by general extension primer D′.

FIG. 5D shows hybridization of primer A with no fluorescent tag to extended product A′-B′-C′-D′ and hybridization of primer F with no fluorescent tag to extended product F′-E′-C′-D′.

FIG. 5E shows amplification of a first target nucleic acid with a first SNP of interest by primer A to produce a first amplified product, and amplification of a second target nucleic acid with a second SNP of interest by primer F, to produce a second amplified product.

FIG. 5F shows a competing reaction to the amplification reactions in FIG. 5E, wherein incorporation and low-efficiency production of a first target nucleic acid with a first SNP of interest is carried out by hybridizable probe A, triggering fluorescence of fluorophore 1 in a first product, thereby allowing detection of a first amplified target nucleic acid, and wherein incorporation and low-efficiency production of a second target nucleic acid with a second SNP of interest is carried out by hybridizable probe F, triggering fluorescence of fluorophore 2 in a second product, thereby allowing simultaneous detection of a second amplified target nucleic acid.

FIG. 6 shows a typical high-density sample array of through-holes according to the prior art.

### DETAILED DESCRIPTION OF SPECIFIC EMBODIMENTS

Definitions. As used in this description and the accompanying claims, the following terms shall have the meanings indicated, unless the context otherwise requires:

“Target nucleic acid,” “target nucleic acid sequence” or “potential target nucleic acid sequence” means any prokaryotic or eukaryotic DNA or RNA including from plants, animals, insects, microorganisms, etc. It may be isolated or present in samples which contain nucleic acid sequences in addition to the target nucleic acid sequence to be amplified. The target nucleic acid sequence may be located within a nucleic acid sequence which is longer than that of the target sequence. The target nucleic acid sequence may be obtained synthetically, or enzymatically, or can be isolated from any organism by methods well known in the art. Particularly useful sources of nucleic acid are derived from tissues or blood samples of an organism, nucleic acids present in self-replicating vectors, and nucleic acids derived from viruses and pathogenic organisms such as bacteria and fungi. Also particularly useful are target nucleic acid sequences which are related to disease states, such as those caused by chromosomal rearrangement, insertion, deletion, translocation and other mutation, those caused by oncogenes, and those associated with cancer.

“Selected” means that a target nucleic acid sequence having the desired characteristics is located and probes are constructed around appropriate segments of the target sequence.

“Probe” or “primer” has the same meaning herein, namely, a nucleic acid oligonucleotide sequence which is single-stranded. The term oligonucleotide includes DNA, RNA and PNA.

A probe or primer is “substantially complementary” to the target nucleic acid sequence if it hybridizes to the sequence under renaturation conditions so as to allow target-dependent ligation or extension. Renaturation depends on specific base pairing between A-X (where X is T or U) and G-C bases to form a double-stranded duplex structure. Therefore, the primer sequences need not reflect the exact sequence of the target nucleic acid sequence. However, if an exact copy of the target sequence is desired, the primer should reflect the exact sequence. Typically, a “substantially complementary” primer will contain at least 70% or more bases which are complementary to the target nucleic acid sequence. More preferably 80% or the bases are complementary, and still more preferably more than 90% of the bases are complementary. Generally, the primer should hybridize to the target nucleic acid sequence at the end to be ligated or extended to allow target-dependent ligation or extension.

Primers may be RNA or DNA and may contain modified nitrogenous bases which are analogs of the normally incorporated bases, or which have been modified by attaching labels or linker arms suitable for attaching labels. Inosine may be used at positions where the target sequence is not known, or where it may be degenerate. The oligonucleotides should be sufficiently long to allow hybridization of the primer to the target sequence and to allow amplification to proceed. They are preferably 15 to 50 nucleotides long, more preferably 20-40 nucleotides long, and still more preferably 25-35 nucleotides longs. The nucleotide sequence of the primers, both content and length, will vary depending on the target sequence to be amplified.

It is contemplated that a primer may comprise one or more oligonucleotides which comprise substantially complementary sequences to the target nucleic acid sequence. Thus, under less stringent conditions, each of the oligonucleotide primers would hybridize to the same segment of the target sequence. However, under increasingly stringent conditions, only that oligonucleotide primer which is most complementary to the target nucleic acid sequence will hybridize. The stringency of the hybridization conditions is generally known to those in the art to be dependent on temperature, solvent, ionic strength, and other parameters. One of the most easily controlled parameters is temperature and since conditions for selective ligation and amplification are similar to those for PCR reactions, one skilled in the art can determine the appropriate conditions required to achieve the level of stringency desired.

Primers suitable for use in the present invention may be derived from any method known in the art, including chemical or enzymatic synthesis, or by cleavage of larger nucleic acids using non-specific nucleic acid-cleaving chemicals or enzymes, or by using site-specific restriction endonucleases.

In order for the ligase of the present invention to ligate the primers together, the primers used are preferably phosphorylated at their 5′-ends. This may be achieved by any known method in the art, including use of T4 polynucleotide kinase. The primers may be phosphorylated in the presence of unlabeled or radiolabeled ATP.

The term “four different nucleotide bases” means deoxythymidine triphosphate (dTTP), deoxyadenosine triphosphate (dATP), deoxycytidine triphosphate (dCTP), and deoxyguanosine triphosphate (dGTP) when the context is DNS, and means uridine triphosphate (UTP), adenosine triphosphate (ATP), cytidine triphosphate (CTP), and guanosine triphosphate (GTP) when the context is RNA. Alternatively, dUTP, dITP (deoxyinosine triphosphate), rITP (riboinosine triphosphate) or any other modified base may replace any one of the four nucleotide bases or may be included along with the four nucleotide bases in the reaction mixture so as to be incorporated into the amplified strand. The amplification steps are conducted in the presence of at least the four deoxynucleoside triphosphates (dATP, dCTP, dGTP and dTTP) or a modified nucleoside triphosphate to produce a DNA strand, or in the present of the four ribonucleoside triphosphates (ATP, CTP, DTP and UTPO or a modified ribonucleoside triphosphate to produce an RNA strand from extension of the primer.

The term “adequate detection of desired amplified product” means detection of at least a two-fold increase in desired amplified target strand over competing linear products.

The term “target sequence detectable above linearly amplified product” means that target sequence is amplified at least two-fold over that of competing linearly amplified non-ligated primer product.

The term “random sequence” as used herein means a sequence unrelated to the target sequence or chosen not to bind to the target sequence or other sequences that might be expected to be present in a test sample.

The term “biocompatible material” as used herein means that the material does not prevent biological processes, such as enzymatic reactions, from occurring when the biocompatible material is present, does not eliminate biological activity or required secondary, tertiary or quaternary structure of biomolecules, such as nucleic acids and proteins, and in general, is not incompatible with biological processes and molecules.

The term “first and second primers being ligatable upon binding to the nucleic acid target sequence” as used herein, means that the first and second primers bind potential target nucleic acid with the 3′-end of the first primer adjacent to, or within about a one- to four-nucleotide gap of, the 5′-end of the second primer, such that subjecting the hybridized first and second primers to appropriate enzymatic or non-enzymatic ligation conditions, including optionally adding a polymerase activity to fill in the gap, allows the first and second primers to be enzymatically or non-enzymatically ligated into a single ligated nucleic acid product.

The term “polymerase” as used herein, means any oligomer synthesizing enzyme, including polymerases, helicases, and other protein fragments capable of polymerizing the synthesis of oligomers.

The term “controlled-temperature reaction mixture” as used herein means, any reaction mixture wherein temperature is controlled by means of a thermocycle apparatus, an isothermal apparatus, or any other means known to allow temperature control of a reaction, including temperature-controllable environments such as water, oil and sand baths, incubation chambers, etc.

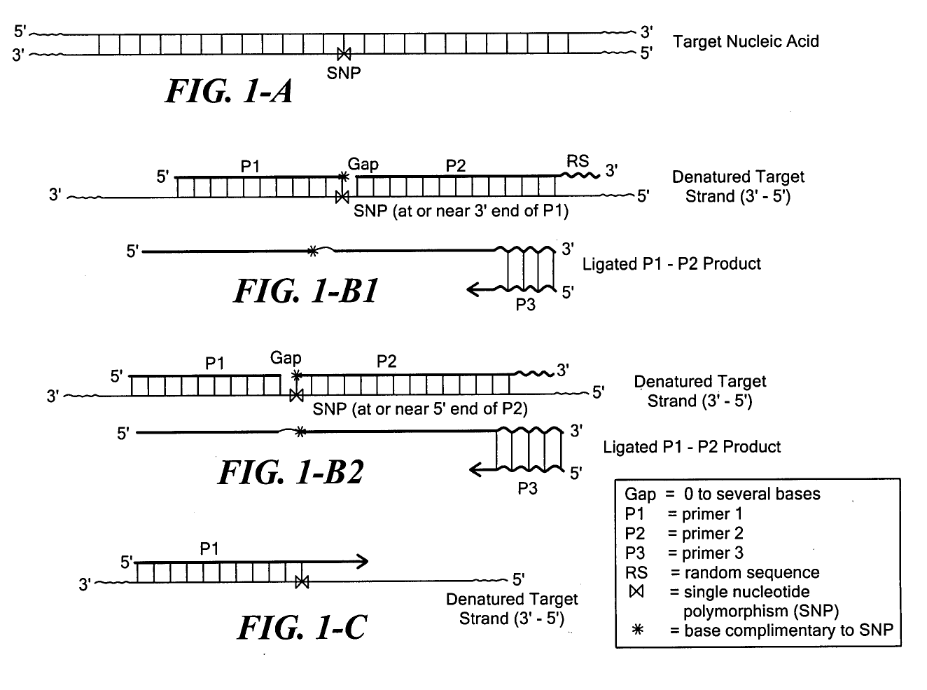

The general assay for identifying single-nucleotide polymorphisms (SNPs) that are not at an end of a target sequence through detection of amplified target sequences, using a dye specific for binding to double-stranded DNA that fluoresces upon binding target sequence according to the present invention, is described below and illustrated in FIGS. 1-5. The assay can be performed in a single-reaction chamber or container, in a series of reaction chambers or containers, in a nanoliter sampling array having a high-density microfluidic array of hydrophilic through-holes, or in a kit comprising such an array plus necessary reagents. Detection may be homogeneous, and may employ a polymerase that lacks 5′ to 3′ exonuclease activity, or a polymerase that lacks 3′ to 5′ exonuclease activity, or a polymerase that lacks both exonuclease activities.

The assay can be done with three (P1, P2, P3) or more (A-B, C-D, F-E, D′) primers, and is able to detect one or more SNPs in a single target simultaneously. In some versions of the assay, the nucleotide complementary to the SNP of the target nucleotide is present at or near the 5′-end of the second primer P2. In other versions, the nucleotide complementary to the SNP of the target nucleotide is present at or near the 3′-end of the first primer P1. In other versions, there are more than one first primers and second primers, these first and second primers designed to generate amplified target sequences having different melting temperatures, such that the assay is able to distinguish individual amplified target sequences because of their individual, and distinct, Tₘs.

Assays may be done with first and second primers that contain degenerate base-pairing positions which allow hybridization of variable regions in target sequences adjacent to the SNP, in this way expanding the general flexibility and utility of the assay.

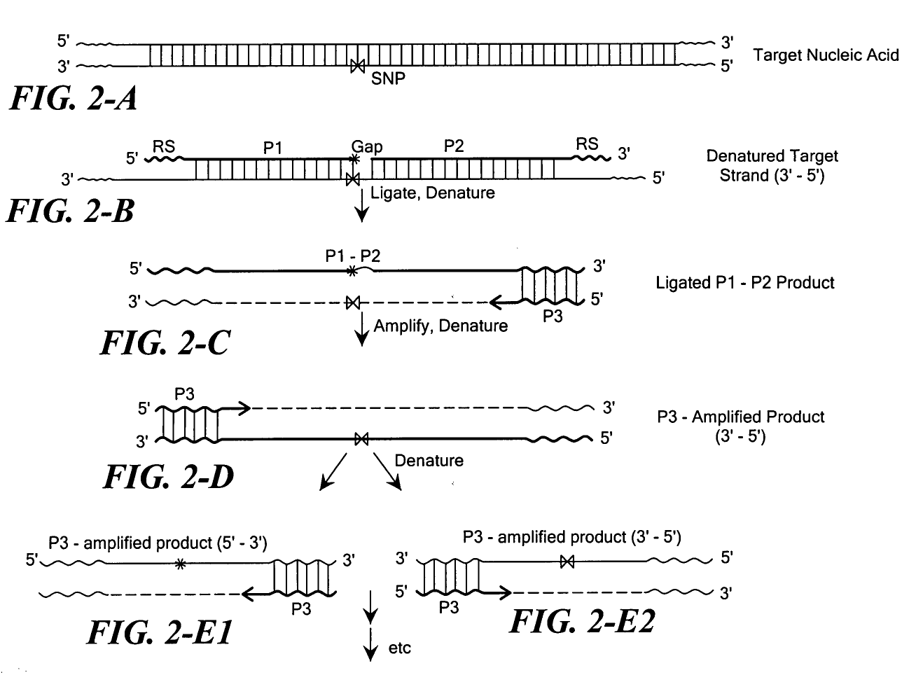

Primers 1 and 2, corresponding to 5′ and 3′ ligation primers, respectively, may be fully or partially complementary to the target sequence. Primer 3 is a generic primer complementary to a random sequence (RS) located at the ends of primers 1 and/or 2 (see FIGS. 1 and 2). The 3′ end of primer 1 and the 5′ end of primer 2 can hybridize either immediately adjacent to each other on the target sequence or can hybridize on the target sequence with a separation, or gap, or one or more nucleotides between them (see FIGS. 1-2 and 4-5). Primers 1 or 2 contain a variant base at or near the 3′ end (P1) or the 5′ end (P2) to enable the primers to bind to SNPS in a target sequence (see FIGS. 1-2). There is also a 3′-hydroxyl group on P2, to facilitate enzymatic or non-enzymatic ligation between P1 and P2 or polymerase extension prior to ligation (to fill in any gap). In addition, the 5′-end of P2 can be modified to prevent undesirable ligation to fragments other than P1.

Similarly, the 5′-end of P1 is phosphorylated to facilitate ligation with P2, and the 3′ end of P1 may be modified to prevent ligation to fragments other than P2. Amplification of target nucleic acid is illustrated in FIGS. 1 and 2. Temperature is used to denature and anneal target nucleic acid and primers, as required, to allow selective extension of ligation of primers P1 and P2.

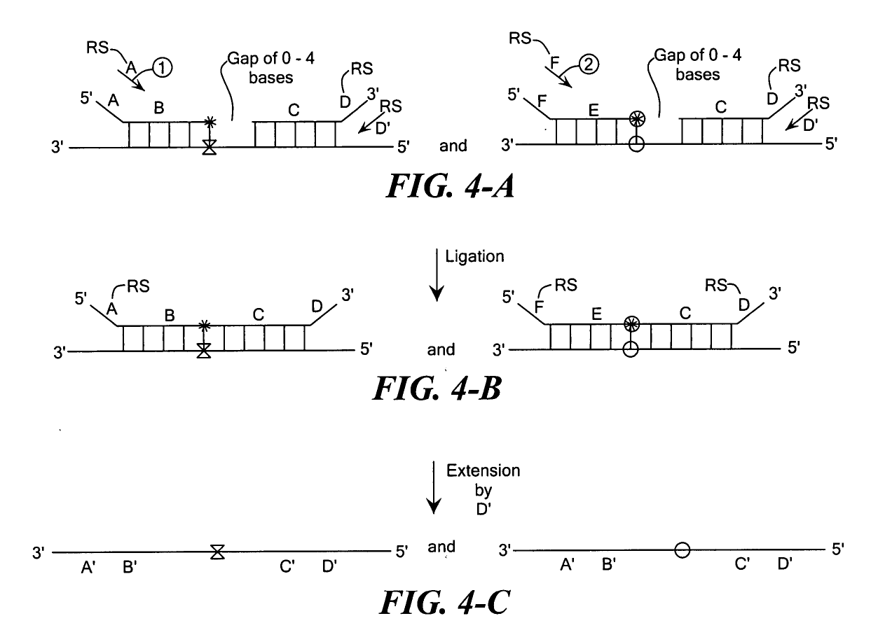

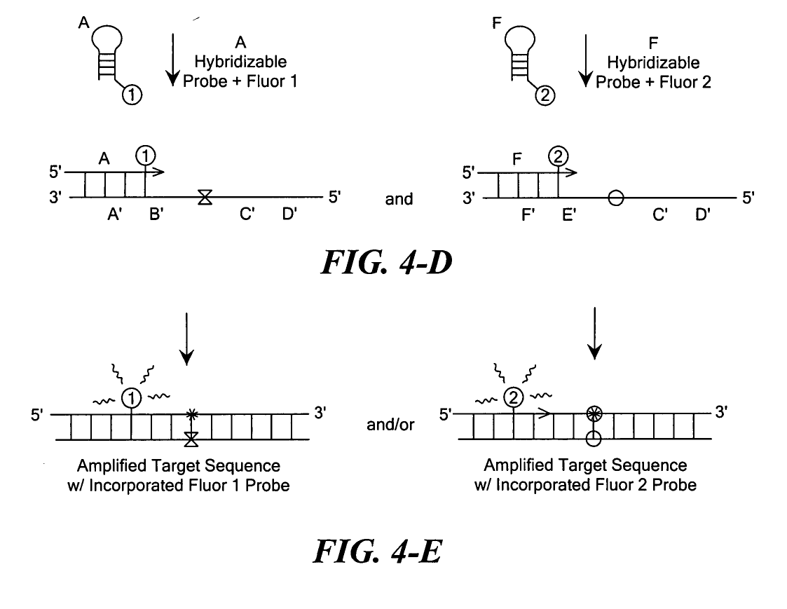

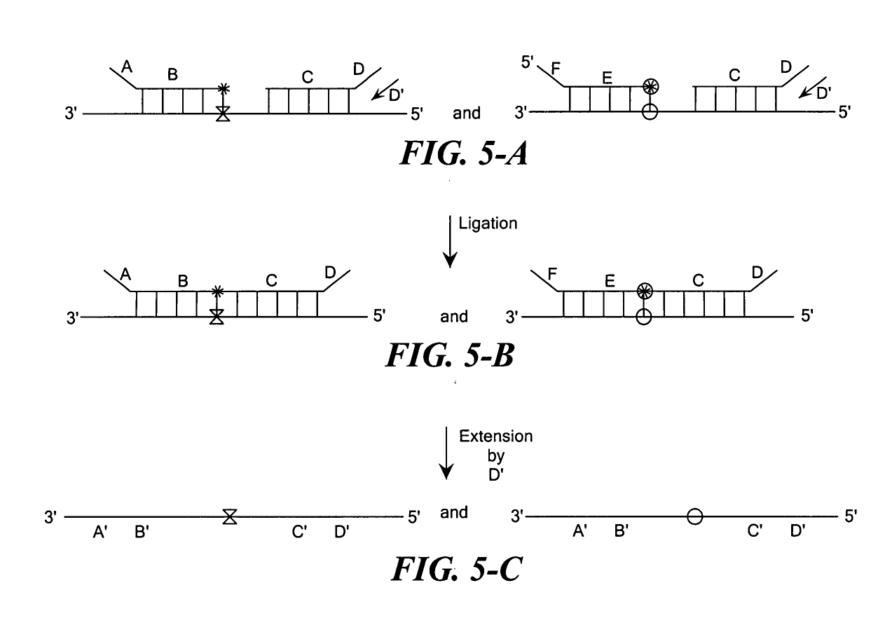

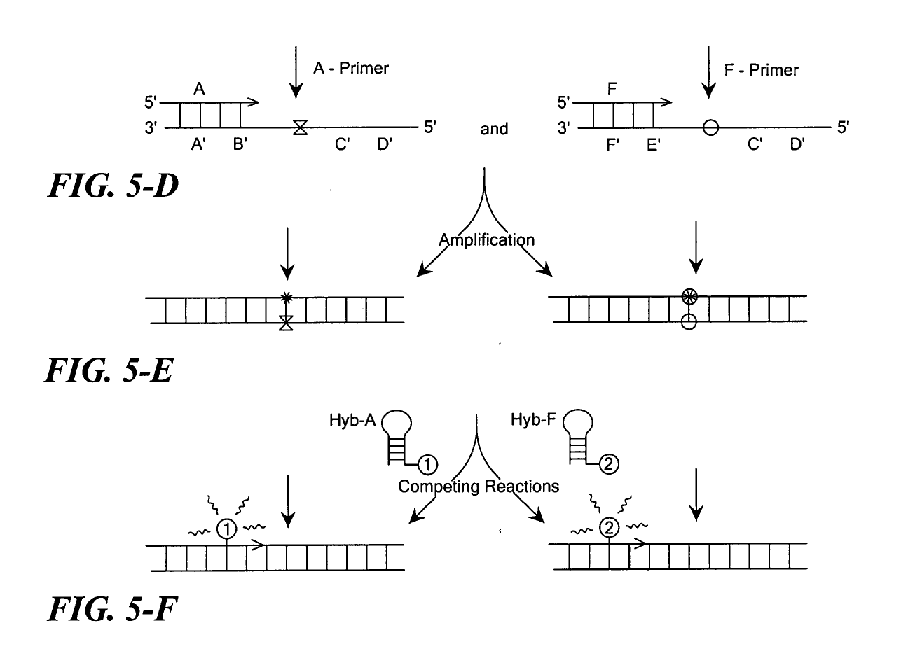

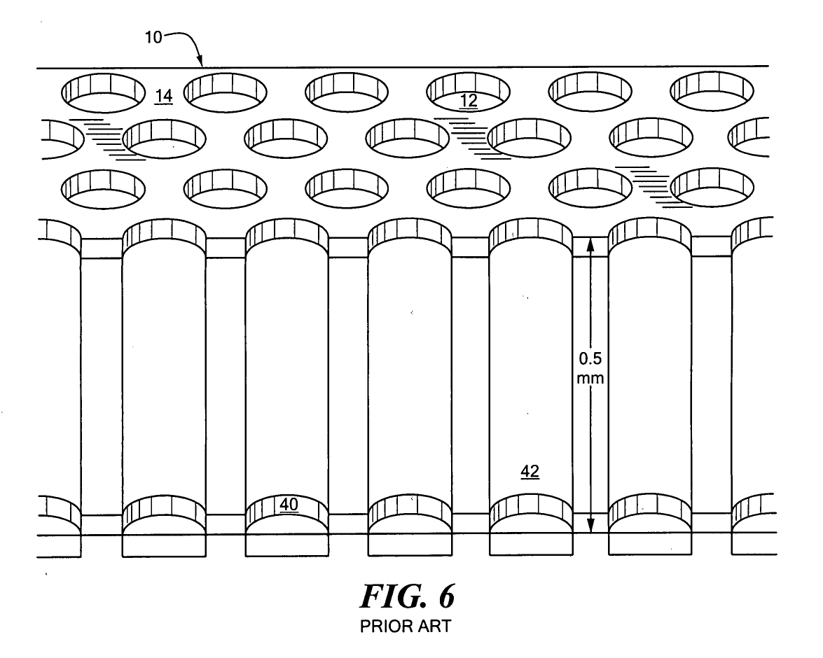

Detection of single-stranded ligation product is carried out using several strategies, some employing a dye specific for binding to double-stranded DNA that is generated either using hybridization probes which hybridize to single-stranded amplified product, or generated after extension and amplification of both the sense and non-sense strands of the ligation product. Other detection strategies employ molecular beacons attached to hybridizable probes. And still other detection strategies employ the use of FRET pairs on hybridizable probes. In some assays, the fluorescent dye is merely added to the reaction mixture, and change in fluorescence intensity is monitored to detect ligated product. In other assays, hybridizable probes are added after generation of ligation product which are specific to the ligation product, and which also contain a molecular beacon, or a fluorescent group and a fluorescence-modifying group. The hybridizable probe may bind to extended ligation product, remaining quenched by the fluorescence-modifying group until extended into amplified product, whereupon the fluorescent group fluoresces and amplified target sequence is detected (see FIG. 4), or the hybridizable probe may be specific for hybridizing across the ligation junction, wherein the probe is again quenched until after hybridizing (see FIG. 5). In the assay illustrated in FIG. 4, one or more hybridizable probes may be used, each having a distinct fluorophore and unique sequence that hybridizes to and amplifies each of one or more target nucleic acid sequences, thereby allowing multiple SNPs to be detected in a single-tube reaction system.

Any of the assays may also be carried out in a nanoliter sampling array. The nanoliter array may comprise one or more platens having at least one hydrophobic surface and a high-density microfluidic array of hydrophilic through-holes. The inner surfaces of the through-holes may be coated with a biocompatible material such as a wax-like polyethylene glycol material, or other biocompatible material. Primers may be applied into the through-holes and then dried, either before or after application of the biocompatible material coating, thereby affixing the primers on, within or under the biocompatible material. Target nucleic acids and reagents for processes used in the selective ligation and amplification assay can be loaded in liquid form into the sample through-holes using capillary action, with typical volumes of the sample through-holes being in the range of from 0.1 picoliter to 1 microliter. The interior surfaces of the through-holes may also have a hydrophilic surface or be coated with a porous hydrophilic material, or as described above, be coated with a biocompatible material such as PEG, to enhance the drawing power of the sample through-holes, attract liquid sample and aid in loading.

Kits for performing the assay may also be prepared, comprising one or more sample platen as described, the primers being affixed within the hydrophilic sample through-holes of the microfluidic array, and also comprising reagents required for the selective ligation and amplification assay. Target nucleic acid sequence(s) can then be added as desired to perform the assay. If not already provided with the kit, enzymes required to carry out the ligation and amplification reactions can also be added along with the target nucleic acid sequence(s).

### EXAMPLES

### Example 1

### Homogeneous Detection of Amplified Target Sequence

Homogeneous detection of amplified target sequences may be carried out using a dye specific for binding to double-stranded DNA or RNA. Primers P1 and P2, upstream and downstream primers, respectively, do not participate in amplification of target sequence, but rather, are responsible for identifying the target sequence containing a SNP. When either primer P1 or P2 contains a match to the SNP of interest in the target sequence, ligation of P1 and P2 occurs, and then primer P3, the general extension primer, amplifies the P1-P2 product. Consequently, concentrations of primers 1 and 2 are preferably optimized and adjusted to not interfere with exponential amplification of the target sequence such that only linear amplification of competing non-target sequences occurs. Examples of ds-DNA- and/or RNA-specific dyes that may be used include SYBR® Green I and SYBR® Green II, YOYO®-1, TOTO®-1, POPO®-3 (see Appendix A, attached hereto), ethidium bromide (EtBr) and any other dye providing adequate sensitivity and ease of detection of desired amplified product.

In a particular embodiment, a sample target sequence of nucleic acid, optionally containing a single nucleotide polymorphism, is mixed with at least three primers—a first upstream primer having at least a portion of its 3′-end substantially complementary to a first segment at a first end of the target sequence, a second downstream primer having at least a portion of its 5′-end substantially complementary to a second segment at a second end of the target sequence, the 5′-end of the second primer being adjacent to the 3′-end of the first primer wherein a nucleotide complementary to the SNP of the target sequence is present at either the 3′-end of the first primer or at the 5′-end of the second primer, and a third general extension primer that is substantially complementary to a random sequence segment at the 3′-end of the second primer and to a substantially similar sequence at the 5′-end of the first primer. Additionally, at least four different nucleotide bases, a thermostable polymerase and a thermostable ligase are included in the reaction mixture, the thermostable polymerase preferably one that lacks 5′ to 3′ exonuclease activity, such as the Stoffel Fragment (see Appendix B, attached hereto). Examples of other thermostable polymerases which lack 5′ to 3′ exonuclease activity include Isis™ DNA polymerase, Pyra™ exo(−) DNA polymerase, and Q-BioTaq DNA polymerase (see Appendix C, attached hereto). Alternatively, the assay may use a thermostable polymerase that lacks 3′ to 5′ exonuclease activity, or a thermostable polymerase that lacks both 5′ to 3′ and 3′ to 5′ exonuclease activity. Examples of thermostable polymerases which lack 3′ to 5′ exonuclease activity include Taq polymerase, SurePrime™ Polymerase, and Q-BioTaq™ DNA polymerase (id.). An example of a thermostable polymerase which lacks both 5′ to 3′ and 3′ to 5′ exonuclease activity is Q-BioTaq DNA polymerase (id.). Addition of a dye specific for ds-DNA such as SYBR® Green I, or specific for RNA such as SYBR® Green II, allows detection of amplified product, by monitoring fluorescence emission of dye-bound nucleic acid product at ˜520 nm (see Appendix D, attached hereto).

As can be seen in FIG. 1-A, a target nucleic acid may contain a SNP within the target sequence. Upon denaturation, Primer 1 (P1) and Primer 2 (P2) bind to the 3′ to 5′ strand of the target sequence, adjacent to the SNP. There may be a gap of several (approximately 2-4) bases between the 3′-end of P1 and the 5′-end of P2, or there may be no gap. In FIG. 1-B1, the base complementary to the SNP of the target sequence is at the 3′-end of P1. Alternatively, the base complementary to the SNP of the target sequence may be at the 5′-end of P2, as shown in FIG. 1-B2. The third primer (P3) contains a random sequence (RS) complementary to the random sequence of the 3′-end of P2, such that after ligation of P1 and P2, P3 binds and extends the ligated primer product, thereby amplifying the complementary strand (5′-3′ strand) of the target sequence. As discussed above, a competing reaction may occur, such that primer P3 binds to primer P2 and extends this sequence to produce a linear product based on the P2 sequence. Preferably, concentrations of primers P1 and P2 are adjusted to minimize the competing linear reaction. As shown in FIG. 1-C, un-ligated primer P1 extends the 3′-5′ strand of the target sequence.

In another, preferred embodiment shown in FIG. 2 (A-E), the first primer (P1) also has a random sequence at the 5′-end. When a primer containing the complement to the SNP, either P1 on its 3′-end or P2 on its 5′-end (see FIG. 1-B), binds to the target strand (see FIG. 2-B), primers P1 and P2 are ligated, and the third primer (P3) then binds to the 3′-end of the ligated P1-P2 product and produces the (3′ to 5′) P3-amplified strand (FIG. 2-C). At this point, primer P3 now also binds to the (3′ to 5′) P3-amplified product and produces the other (5′ to 3′) amplified product (see FIG. 2-D). Both target strands have now been produced, and can go on to yield exponentially amplified target sequence (FIGS. 2-E1 and 2-E2).

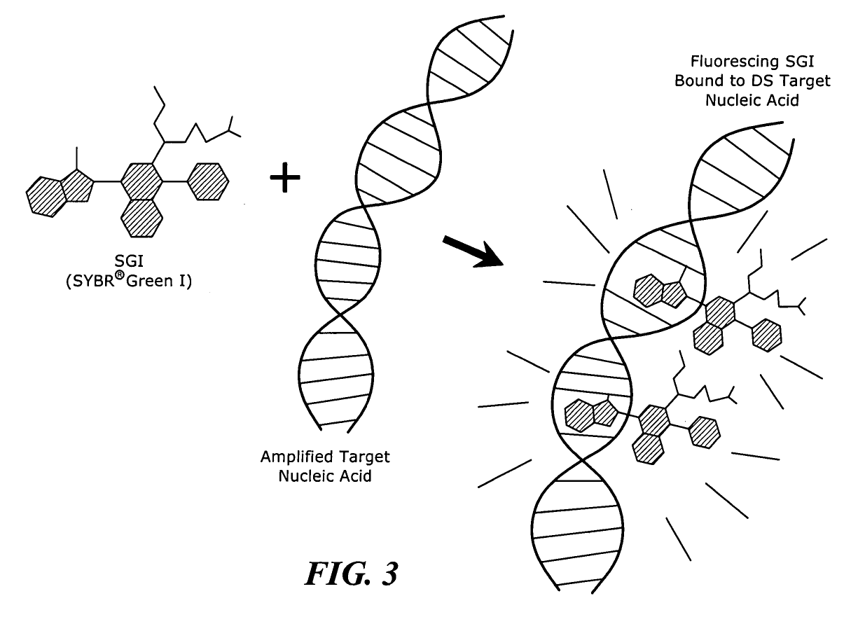

Additionally, detection with a fluorescent dye, such as SYBR® Green I (SGI) may be done at temperatures above the Tₘ of the linear product, i.e., any product produced non-exponentially, thereby removing competing signal from any dye bound to linear product. SYBR® Green I and other dyes that bind to double-stranded nucleic acids do not bind to nucleic acids above their Tₘs because at those elevated temperatures, the nucleic acids are denatured. As seen in the cartoon of FIG. 3, a dye such as SYBR® Green I binds to double-stranded amplified target nucleic acid with a concomitant large increase in fluorescence. Although SGI is shown in FIG. 3 as intercalating into the amplified target ds-nucleic acid, nothing in the figure is intended to suggest either an actual structure, or actual mode of binding, for SGI with ds-nucleic acids.

Alternatively, the use of molecular beacon probes, having a fluorescent group on one end and a fluorescence-quenching group on the other, may be used. In this system, the molecular beacon remains quenched until being bound to amplified product (see, for example, Appendix E, attached hereto) because the molecular probe is typically in a hairpin conformation with the fluorescent group in close proximity to the fluorescence-quenching group, until the probe binds to the target amplified product (causing the hairpin structure to unfold, separating the fluorescent group from the quenching group). Examples of fluorescence-quenching groups appropriate for embodiments of the present invention include the dark quencher dabcyl, and the Eclipse™ Quencher from Epoch (id.). Examples of appropriate fluorescent groups that may be used in accordance with the present invention include Epoch's Yakima Yellow™ and Redmond Red™ (id.), and any other appropriate fluorescent dye whose fluorescence may be quenched to an appropriately positioned quencher molecule.

In another embodiment, real-time amplification may be measured using a TaqMan® probe that is homologous to an internal sequence of the target nucleic acid, and having a fluorogenic 5′-end and a quencher 3′-end. During PCR amplification and extension, the quencher molecule is removed from the probe by 5′-exonuclease activity, releasing the fluorescent reporter molecule from close proximity to the quencher molecule on the 3′-end of the probe, thereby producing an increase in fluorescence emission as amplified product is produced (see Appendices F and G, attached hereto). In this system, a polymerase having 5′ to 3′ exonuclease activity is required.

Another embodiment utilizes a detection method for real-time amplification measurement that involves the use of a pair of amplification primers, one of which matches the random sequence of primer 2. One of these primers in the pair is labeled with a fluorescent group that only fluoresces when incorporated into a PCR product, similar to Lux™ primers known in the art (see Appendix H, attached hereto). In such an embodiment, the fluorescent group is quenched by secondary structure before incorporation into double-stranded product, such that prior to incorporation, a sequence in the primer/probe binds to a complementary sequence in the primer/probe containing the fluorescent group, quenching the fluorescent group. In another embodiment, primers 1 and 2 are FRET partners, such that when hybridized to the amplified target sequence, produced only after primers 1 and 2 are ligated and amplified, they fluoresce (see Appendices E and also A) and thus permit detection of amplified target sequence. In a preferred embodiment, fluorescence detection would be carried out above the either the Tₘ for primer P1, or above the Tₘ for primer P2, or alternatively be carried out above the Tₘs of both primers P1 and P2, to avoid background signal from possible hybridization of P1 and/or P2 to amplified target.

In another embodiment, primer may be designed to exponentially amplify target nucleic acid products that are distinguishable by an increase or decrease in melting temperature (Tₘ), wherein the exponentially amplified target sequence is either stabilized as indicated by an increase in Tₘ or de-stabilized, as indicated by a decrease in Tₘ, relative to the melting temperatures of linearly produced non-target product produced from non-ligated primers. Variability in the random sequence, or elsewhere in the primers, may be used to produce such exponentially amplified target nucleic acid sequence distinguishable by melting temperature from the linear product.

In another embodiment, a probe specific for hybridizing across the ligation junction formed after ligation of the first and second primers may be used. Such a probe may have a hairpin conformation with a fluorescent reporter group on one end and a fluorescence-quenching group on the other end whereby no fluorescence occurs when the probe is not bound across the ligation junction. By optimizing reaction conditions, such as temperature and/or ionic strength, the hairpin would be stabilized by binding across the ligation junction, whereupon fluorescence would occur and emission could be monitored to detect amplified product.

### Example 2

### Single-Tube Reaction System for Distinguishing SNPs

One preferred embodiment of the present invention is the single-tube reaction system shown in FIG. 4. Similar to the embodiments shown in FIGS. 1 and 2 and discussed above in Example 1, a three-primer system is utilized to identify a SNP of interest in a target sequence of nucleic acid. Again, there is an upstream primer and a downstream primer that bind to the target nucleic acid, flanking the SNP of interest. The 3′-end of the upstream primer may be directly adjacent to the 5′-end of the downstream primer, or there may be a gap of between about 1 to 4 bases between the 3′-end of the upstream primer and the 5′-end of the downstream primer. Either the 3′-end of the upstream primer or the 5′-end of the downstream primer may contain the complement to the SNP of interest in the target nucleic acid.

Unlike the embodiments shown in FIGS. 1 and 2, however, the single-tube reaction system allows simultaneous single-tube identification and distinction between one or more SNPs of interest in one or more target nucleic acid sequences of interest. This is accomplished by using unique sequences in each of the random sequence regions of the upstream primer and the downstream primer (the two which ligate) and the general extension primer. As see in FIG. 4A, a single-tube reaction system may contain a first upstream primer A-B with random sequence A, which identifies a first SNP of interest in a first target nucleic acid segment, and a second upstream primer F-E with random sequence F, which identifies a second SNP or interest in a second target nucleic acid segment, and a general extension primer with random sequence D′ complementary to random sequence D present in downstream primer C-D, wherein C is common to both target nucleic acid segments.

Upon successful identification and binding to a target nucleic acid having a SNP of interest, upstream primers A-B and/or F-E will be ligated to downstream primer C-D, creating ligation products A-B-C-D and/or F-E-C-D. If a gap is present between the 3′-end of the upstream primer and the 5′-end of the downstream primer, the gap will first be filled in by a polymerase activity, followed by ligation to form the ligation products. Extension of both ligation products can then occur by general extension primer D′, to produce extended products A′-B′-C′-D′ and F′-E′-C′-D′.

Next, hybridizable probe A with fluorophore 1 and hybridizable probe F with fluorophore 2, hybridize to extended products A′-B′-C′-D′ and F′-E′-C′-D′, respectively, which is followed by amplification such that each of the probes with its particular fluorescent tag is incorporated into amplified product (A-B-C-D or F-E-C-D), triggering fluorescence of either fluorophore 1 or fluorophore 2 or both. In this way, one or more SNPs may be identified and distinguished in a single-tube reaction system by monitoring the fluorescent signals of the two (or more) fluorophores upon incorporation into amplified product.

In another embodiment, an alternative single-tube reaction system for identifying and distinguishing one or more SNPs in one or more target nucleic acid segments is shown in FIGS. 5A-5F. FIGS. 5A through 5C are identical to FIGS. 4A through 4C, in that upstream primers A-B and F-E, downstream primer C-D, and general extension primer D′ are present in the single-tube reaction system. Again, either the 3′-end of the upstream primers may contain the complement to the SNP of interest in the target nucleic acids, or the 5′-end of the downstream primer may contain the complement to the SNP of interest in the target nucleic acids, and upon binding to the target nucleic acids, the two primers may be adjacent, or have a gap of about 1-4 bases between the 3′-end of the upstream primer and the 5′-end of the downstream primer, which must be filled by a polymerase, before ligation between the upstream and downstream primer can occur.

As shown in FIG. 5D, however, the alternative single-tube reaction system does not use hybridizable probes A and F with fluorophores 1 and 2 to amplify target nucleic acid, but rather, uses regular primers A and F to amplify extended products A′-B′-C′-D′ and F′-E′-C′-D′ into amplified target nucleic acids products A-B-C-D or F-E-C-D. Such a system may be advantageous when a particular target nucleic acid does not amplify efficiently with hybridizable probes that have bulky fluorophores attached to them. In this alternative single-tube reaction system, the amplified target nucleic acids are detected after amplification, by additional fluorescent-tagged hybridizable probes hyb-A and hyb-F, which differ from regular primers A and F in that they are shorter, and have secondary structure that dissolve at lower temperatures than the annealing temperatures of primers A and F (or fluorescent probes A and F in FIG. 4). This allows inefficient competition between hyb-A and hyb-F probes and regular primers A and F, in amplification of extended products A′-B′-C′-D′ and F′-E′-C′-D′ into target nucleic acid products A-B-C-D or F-E-C-D, but allows enough competing reaction to occur to measure fluorescence of fluorophores 1 and 2, thereby allowing detection and quantitation of amplified target nucleic acid product.

Although use of a general extension primer such as D′ that is complementary to a sequence D in segment C-D common to both target nucleic acid segments is convenient in the single-tube reaction systems described above and exemplified in FIGS. 4 and 5, it is not required. It is envisioned that single-tube reaction systems could also be adapted for creating ligation products with with A-B and F-E using more than one extension primer simultaneously. The selectivity of the first primer A-B for the first SNP and the second primer E-F for the second SNP will ensure selective ligation, even with additional primers being used to generate the C-X product to be ligated.

Upon successful identification and binding to a target nucleic acid having a SNP of interest, upstream primers A-B and/or F-E will be ligated to downstream primer C-G and C-H, respectively, creating ligation products A-B-C-G and/or F-E-C-H. If a gap is present between the 3′-end of the upstream primer and the 5′-end of the downstream primer, the gap will first be filled in by a polymerase activity, followed by ligation to form the ligation products. Extension of both ligation products can then occur by extension primers G′ and H′, to produce extended products A′-B′-C′-G′ and F′-E′-C′-H′.

As described above, one or more SNPs may be identified and distinguished in a single-tube reaction system by a) monitoring the fluorescent signals of two (or more) fluorophores upon incorporation into amplified product, or b) detecting fluorescent signals after amplification, by use of additional fluorescent-tagged hybridizable probes hyb-A and hyb-F.

### Example 3

### A Nanoliter Sampling Array

Another embodiment of the present invention encompasses a nanoliter sampling array. Any array presently available in the prior art may be used, but an array of particular utility, similar to that described in U.S. Provisional Application Ser. No. 60/518,240, filed Nov. 7, 2003, and U.S. regular application Ser. No. 10/984,027 filed on Nov. 8, 2004, both of which are hereby incorporated by reference herein, is one preferred array. In this particular embodiment, the array comprises a first platen having at least one hydrophobic surface and having a high-density microfluidic array of hydrophilic through-holes. A target nucleic acid sequence is selected, and the array is prepared wherein each through-hole in the array contains at least a first primer having at least a portion of its 3′-end substantially complementary to a first segment at a first end of the nucleic acid target sequence and a second primer having at least a portion of its 5′-end substantially complementary to a second segment at a second end of the nucleic acid target sequence, the 5′-end of the second primer being adjacent to the 3′-end of the first primer upon binding to the potential nucleic acid target sequence. FIG. 4 shows such an array, known in the prior art. Array chip 10 typically may be from 0.1 mm to more than 10 mm thick; for example, from 0.3 to 1.52 mm thick, and commonly 0.5 mm. Typical volumes of the sample through-holes 12 could be from 0.1 picoliter to 1 microliter, with common volumes in the range of 0.2 to 100 nanoliters, for example, about 35 nanoliters: Capillary action or surface tension of the liquid samples may be used to load the sample through-holes 12. For typical chip dimensions, capillary forces are strong enough to hold liquids in place. Chips loaded with sample solutions can be waved in the air, and even centrifuged at moderate speeds, without displacing the samples.

To enhance the drawing power of the sample through-holes 12, the target area of the receptacle interior walls 42 may have a hydrophilic surface that attracts a liquid sample. Alternatively, the sample through-holes 12 may contain a porous hydrophilic materiel that attracts a liquid sample. In some embodiments, the sample through-holes in the array may be coated with a biocompatible material such as polyethylene glycol, and the primers may be affixed on, within or under the biocompatible material on the surface of the through-holes by drying the primers after application to the through-holes. To prevent cross-contamination (crosstalk), the exterior planar surfaces 14 of chip 10 and a layer of material 40 around the openings of sample through-holes 12 may be of a hydrophobic material. Thus, each sample through-hole 12 has an interior hydrophilic region bounded at either end by a hydrophobic region.

The through-hole design of the sample through-holes 12 avoids problems of trapped air inherent in other microplate structures. This approach, together with hydrophobic and hydrophilic patterning enable self-metered loading of the sample through-holes 12. The self-loading functionality helps in the manufacture of arrays with pre-loaded reagents, and also in that the arrays will fill themselves when contacted with an aqueous sample material.

### Example 3

### Method for Identifying a SNP in a Target Sequence of Nucleic Acid

Yet another embodiment is a method for identifying a single nucleotide polymorphism (SNP) in a target sequence of nucleic acid. A target sequence of nucleic acid is identified, and primers are prepared according to standard methods, such that two primers, P1 and P2, are designed to flank an internally-positioned SNP on one strand of the target nucleic acid sequence and are designed to be ligated with a thermally stable ligase. Primer P1 and P2 are further designed such that the base complementary to the SNP in the target sequence is either on the 3′-end of P1, or on the 5′-end of P2. In this particular method, a nanoliter sampling array is used. The method comprises providing a first platen having a high-density microfluidic array of through-holes is provided wherein each through-hole of the array contains a first primer having at least a portion of its 3′-end substantially complementary to a first segment at a first end of the target sequence, and a second primer having at least a portion of its 5′-end substantially complementary to a second segment at a second end of the target sequence. Upon binding to the target sequence, the 5′-end of the second primer is adjacent to the 3′-end of the first primer.

The method further comprises introducing a sample containing the target nucleic acid sequence with internal SNP into the array, and introducing reagents into the through-holes in the array wherein the reagents include a third primer having a random sequence capable of amplifying ligated primer P1-P2 product, a thermostable polymerase, a thermostable ligase, and at least four different nucleoside triphosphates. Additional steps in the method comprise thermocycling the array with primers, target nucleic acid, and reagents, and detecting the resulting amplified target nucleic acid sequence. Optionally, the thermostable polymerase may lack 5′ to 3′ exonuclease activity, or it may lack 3′ to 5′ exonuclease activity, or it may lack both 5′ to 3′ and 3′ to 5′ exonuclease activity.

It is also envisioned that the detecting step may comprise the use of a dye specific for binding to double-stranded DNA or to RNA that fluoresces upon binding amplified target sequence. Suitable dyes include SYBR® Green I, SYBR® Green II, YOYO®-1, TOTO®-1, POPO®-3, EtBr, and any other dye capable of providing low-sensitivity detection of amplified target sequence by fluorescence emission.

Alternatively, detection may occur through the addition of probes specific for hybridization across the ligation junction of the ligated P1-P2 primer product, where such probes contain a fluorescent group and a fluorescence-modifying group such as a fluorescence quencher.

In another alternative embodiment, detection may involve the use of a probe containing a fluorescent group and a fluorescence-modifying group such as a fluorescence quencher that is specific for hybridizing to a region of the target sequence. In this particular embodiment, the fluorescence-modifying group is excised upon extension of the probe, and the fluorescent group thus fluoresces, allowing detection of amplified product.

Additional embodiments of the present invention include a kit for use in identification of amplified target nucleic acid sequences, wherein the kit provides a sample platen having one hydrophobic surface and having a high-density microfluidic array of hydrophilic through-holes. In one particular kit each through-hole contains at least a first primer having at least a portion of its 3′-end substantially complementary to a first segment at a first end of potential nucleic acid target sequence, a second primer having at least a portion of its 5′-end substantially complementary to a second segment at a second end of the potential nucleic acid target sequence, the 5′-end of the second primer being adjacent to the 3′-end of the first primer upon binding to the potential nucleic acid target sequence and a reagent platen having a high-density microfluidic array of through-holes with each through-hole containing a third primer that is substantially complementary to a random sequence segment at the 3′-end of the second primer and to a substantially similar sequence at the 5′-end of the first primer, at least four different nucleotide bases, a thermostable ligase and a fluorescent dye. In this particular embodiment, the reagent platen has a structural geometry that corresponds to the sample platen allowing delivery of reagent components and target nucleic acid sample to the primers in the sample platen. In some embodiments of the kit, the primers may be affixed on, within or under a biocompatible material such as a wax-like coating in the through-holes by drying the primers after being applied to the through-holes, wherein the biocompatible material may comprise, for example, a polyethylene glycol (PEG) material. To perform the selective ligation and amplification reaction for identification of an amplified target nucleic acid sequence, the user would merely add a sample containing the target nucleic acid, a thermostable polymerase, and optionally a buffer supplied with the kit to the through-holes.

1. An improved assay of the type for amplifying a specific target nucleic acid sequence, wherein the target sequence comprises an internal SNP of interest, the assay being a selective ligation and amplification method of the type using a controlled-temperature reaction mixture including the target sequence, ligatable first and second primers having at least a portion substantially complementary to first and second segments of the target sequence, respectively, and a third primer that is substantially complementary to a random sequence segment of the first and second primers, wherein the improvement comprises:
homogeneously detecting amplified target sequence using a dye specific for binding to double-stranded (ds) DNA that fluoresces upon binding target sequence.

2. An improved assay according to claim 1 wherein a nucleotide complementary to the SNP of the target sequence is present at the 5′-end of the second primer.

3. An improved assay according to claim 1, wherein the dye comprises SYBR® Green.

4. An improved assay according to claim 1, wherein the assay further comprises:
using a first primer and a second primer at concentrations such that a ligated product produces exponentially amplified target sequence detectable above linearly amplified non-ligated primer product.

5. An improved assay according to claim 1, wherein the assay further comprises:
using a plurality of first primers and second primers designed to generate amplified target sequences with differential melting curves;
distinguishing individual amplified target sequences by differences in melting temperatures (Tₘs).

6. An improved assay according to claim 1, wherein the first and second primers contain degenerate base-pairing positions to allow hybridization to variable regions in target sequences adjacent to the SNP.

7. An improved assay of the type for amplifying a specific target nucleic acid sequence, wherein the target sequence comprises an internal SNP of interest, the assay being a selective ligation and amplification method of the type using a temperature-controllable reaction mixture including the target sequence, ligatable first and second primers having at least a portion substantially complementary to first and second segments of the target sequence, respectively, and a third primer that is substantially complementary to a random sequence segment of the first and second primers, wherein the improvement comprises:
detecting amplified target sequence using a probe specific for hybridizing across a ligation junction formed between the first primer and second primer after binding to the target sequence wherein the probe specific for hybridizing across the ligation junction contains a molecular beacon.

8. An improved assay according to claim 7, wherein the probe specific for hybridizing across the ligation junction has a fluorescent group and a fluorescence-modifying group.

9. An improved assay according to claim 8, wherein the fluorescent group is quenched when the probe is not bound across the ligation junction and the fluorescent group fluoresces when the probe is bound across the ligation junction.

10. An improved assay of the type for amplifying a specific target nucleic acid sequence, wherein the target sequence comprises an internal SNP of interest, the assay being a selective ligation and amplification method of the type using a temperature-controllable reaction mixture including the target sequence, ligatable first and second primers having at least a portion substantially complementary to first and second segments of the target sequence, respectively, and a third primer that is substantially complementary to a random sequence segment of the first and second primers, wherein the improvement comprises:
detecting amplified target sequence using a probe specific for hybridizing to a region of the target sequence wherein the probe contains a fluorescent group and a fluorescence-modifying group.

11. An improved assay according to claim 10, wherein upon extension of the probe, the fluorescence-modifying group is excised and the fluorescent group fluoresces.

12. An improved assay according to claim 7 or 10, wherein the fluorescent group is quenched before incorporation into double-stranded product and is dequenched after incorporation into double-stranded product.

13. An improved assay according to claim 12, wherein the fluorescent group is quenched by secondary structure before incorporation into double-stranded product, such that before incorporation, a sequence in the probe binds to a complementary sequence in the probe containing the fluorescent group, quenching the fluorescent group.

14. A nanoliter sampling array comprising:
a) a first platen having at least one hydrophobic surface and having a high-density microfluidic array of hydrophilic through-holes;
wherein each through-hole contains
i) a first primer having at least a portion of its 3′-end substantially complementary to a first segment at a first end of a potential nucleic acid target sequence; and
ii) a second primer having at least a portion of its 5′-end substantially complementary to a second segment at a second end of the potential nucleic acid target sequence, the first and second primers being ligatable upon binding to the potential nucleic acid target sequence.

15. A nanoliter sampling array according to claim 14, further comprising:
a second platen having at least one hydrophobic surface and having a high-density microfluidic array of hydrophilic through-holes;
wherein the first and second platen are fixedly coupled such that the through-holes of each are aligned.

16. A nanoliter sampling array according to claim 14, wherein at least one pair of aligned through-holes contains first reagents for a first assay process and second reagents for a second assay process.

17. An array according to claim 16, wherein one of the assay processes is PCR amplification.

18. An array according to claim 16, wherein one of the assay processes is detection of amplified target nucleic acid sequence having a SNP.

19. An array according to claim 18, wherein detection of amplified target nucleic acid sequence comprises using a dye specific for binding to double-stranded (ds) DNA that fluoresces upon binding target sequence.

20. An array according to claim 18, wherein detection of amplified target nucleic acid sequence comprises distinguishing individual amplified target sequences by differences in melting temperatures (Tₘs).

21. An array according to claim 18, wherein detection of amplified target nucleic acid sequence comprises using a probe specific for hybridizing across a ligation junction formed between the first primer and second primer after binding to the target sequence, wherein the probe has a fluorescent group and a fluorescence-modifying group.

22. An array according to claim 18, wherein detection of amplified target nucleic acid sequence comprises using a probe containing a fluorescent group and a fluorescence-modifying group specific for hybridizing to a region of the target sequence wherein upon extension of the probe, the fluorescence-modifying group is excised and the fluorescent group fluoresces.

23. An array according to claim 22, wherein the fluorescent group is quenched before incorporation into double-strand product and is dequenched after incorporation into double-stranded product.

24. An array according to claim 23, wherein the fluorescent group is quenched by secondary structure before incorporation into double-stranded product such that before incorporation, a sequence in the probe binds to a complementary sequence in the probe containing the fluorescent group, quenching the fluorescent group.

25. A nanoliter sampling array according to any of claims 14-24, wherein the primers are affixed on, within or under a coating of the sample through-holes by drying, the coating comprising a biocompatible material.

26. A method of identifying a SNP in a target sequence of nucleic acid, the method comprising:
providing a first sample platen having a high-density microfluidic array of through-holes, each through-hole having a first primer having at least a portion substantially complementary to a first segment of the target sequence, a second primer having at least a portion substantially complementary to a second segment of the target sequence, the 5′-end of the second primer ligatable to the 3′-end of the first primer after binding nucleic acid target sequence, and a third primer that is substantially complementary to a random sequence segment of the first and second primers;
introducing a sample containing a target sequence of nucleic acid having a SNP of interest to the through-holes in the array;
introducing reagents to the through-holes in the array, the reagents including a reagent for effecting amplification, a reagent for effecting ligation, and at least four different nucleotide bases;
effecting ligation of the first and second primers to produce a ligated product;
effecting amplification of the ligated product and target sequence;
detecting amplified target sequence.

27. A method of identifying a SNP in a target sequence of nucleic acid according to claim 26, wherein effecting ligation and effecting amplification comprises addition of a ligase and a polymerase followed by subjecting the array to controlled-temperature conditions.

28. A method according to claim 26 wherein detecting comprises using a dye specific for binding to double-stranded (ds) DNA that fluoresces upon binding target sequence.

29. A method according to claim 26 wherein detecting comprises distinguishing individual amplified target sequences by differences in melting temperatures (Tₘs).

30. A method according to claim 26 wherein detecting comprises using a probe specific for hybridizing across a ligation junction formed between the first primer and second primer after binding to the target sequence, wherein the probe has a fluorescent group and a fluorescence-modifying group.

31. A method according to claim 26 wherein detecting comprises using a probe containing a fluorescent group and a fluorescence-modifying group specific for hybridizing to a region of the target sequence wherein upon extension of the probe, the fluorescence-modifying group is excised and the fluorescent group fluoresces.

32. An improved assay according to claim 30, wherein the fluorescent group is quenched before incorporation into double-strand product and is dequenched after incorporation into double-stranded product.

33. A method according to claim 32, wherein the fluorescent group is quenched by secondary structure before incorporation into double-stranded product, such that before incorporation, a sequence in the probe binds to a complementary sequence in the probe containing the fluorescent group, quenching the fluorescent group.

34. A kit for use in identification of amplified target nucleic acid sequences, the kit comprising:
a) a sample platen having one hydrophobic surface and having a high-density microfluidic array of hydrophilic through-holes;
wherein each sample platen through-hole contains at least
i) a first primer having at least a portion substantially complementary to a first segment of potential nucleic acid target sequence;
ii) a second primer having at least a portion substantially complementary to a second segment of the potential nucleic acid target sequence, the first and second primers ligatable upon binding to the potential nucleic acid target sequence;
b) a reagent platen having a high-density microfluidic array of through-holes, each reagent platen through-hole containing at least
i) a third primer that is substantially complementary to a random sequence segment of the first and second primers;
ii) at least four different nucleotide bases;
iii) a reagent for effecting ligation; and
iv) a fluorescent dye
the reagent platen having a structural geometry that corresponds to the sample platen allowing delivery of reagent components and target nucleic acid sample to the primers in the sample platen.

35. A kit for use in identification of amplified target nucleic acid sequences according to claim 34, wherein a PCR-compatible buffer is also included.

36. A kit according to claim 34, wherein the fluorescent dye comprises SYBR® Green I, SYBR® Green II, YOYO®-1, TOTO®-1, POPO®-3, or ethidium bromide.

37. A kit according to any of claims 34-36, wherein the primers are affixed on, within or under a coating of the sample through-holes by drying, the coating comprising a biocompatible material.

38. An improved assay of the type for amplifying a specific target nucleic acid sequence, wherein the target sequence comprises an internal SNP of interest, the assay being a selective ligation and amplification method of the type using a controlled-temperature reaction mixture including the target sequence, ligatable first and second primers having at least a portion substantially complementary to first and second segments of the target sequence, respectively, and a third primer that is substantially complementary to a random sequence segment of the first and second primers, wherein the improvement comprises:
detecting one or more amplified target sequences in a single-tube reaction system using one or more probes specific for hybridizing to a region of one or more target nucleic acid sequences, wherein the one or more probes each contain a distinct fluorescent group and a fluorescence-modifying group and wherein hybridization of the one or more probes results in fluorescence of the distinct fluorescent group.

39. An improved assay according to claim 38, wherein upon extension of the probe, the fluorescence-modifying group is excised and the fluorescent group fluoresces.

40. An improved assay according to claim 38, wherein the fluorescent group is quenched before incorporation into double-strand product and is dequenched after incorporation into double-stranded product.

41. An improved assay according to claim 40, wherein the fluorescent group is quenched by secondary structure before incorporation into double-stranded product, such that before incorporation, a sequence in the probe binds to a complementary sequence in the probe containing the fluorescent group, quenching the fluorescent group.

42. An improved assay according to claim 38, wherein the one or more target nucleic acid sequences is 2, each having a distinct SNP of interest.

43. An improved assay according to claim 42, wherein the one or more hybridizable probes is 2, each having a distinct fluorophore and unique sequence that hybridizes to and amplifies each of the 2 target nucleic acid sequences.

44. An improved assay according to claim 38, wherein the one or more target nucleic acid sequences is 3, each having a distinct SNP of interest.

45. An improved assay according to claim 44, wherein the one or more hybridizable probes is 3, each having a distinct fluorophore and unique sequence that hybridizes to and amplifies each of the 3 target nucleic acid sequences.

46. An improved assay according to claim 38, wherein the one or more target nucleic acid sequences is 4, each having a distinct SNP of interest.

47. An improved assay according to claim 46, wherein the one or more hybridizable probes is 4, each having a distinct fluorophore and unique sequence that hybridizes to and amplifies each of the 4 target nucleic acid sequences.

48. An improved assay of the type for amplifying a specific target nucleic acid sequence, wherein the target sequence comprises an internal SNP of interest, the assay being a selective ligation and amplification method of the type using a controlled-temperature reaction mixture including the target sequence, ligatable first and second primers having at least a portion substantially complementary to first and second segments of the target sequence, respectively, and a third primer that is substantially complementary to a random sequence segment of the first and second primers, wherein the improvement comprises:
detecting one or more amplified target sequences in a single-tube reaction system using one or more fourth primers, each having a fluorescent group and a fluorescent-modifying group, and each being complementary to a unique region of a ligated template for the one or more target nucleic acid sequences, wherein upon fourth primer incorporation into and amplification of the one or more target nucleic acid sequences, fluorescence of the distinct fluorescent group occurs such that detection of one or more amplified target nucleic acid sequences in a single-tube reaction system results.

49. An improved assay according to any of claims 1, 7, 10, 26, or 48, further comprising using a polymerase that lacks 5′ to 3′ exonuclease activity.

50. An improved assay according to any of claims 1, 7, 10, 26, or 48, further comprising using a polymerase that lacks 3′ to 5′ exonuclease activity.

51. An improved assay according to any of claims 1, 7, 10, 26, or 48, further comprising using a polymerase that lacks 5′ to 3′ exonuclease activity and 3′ to 5′ exonuclease activity.

52. An improved assay according to any of claims 7, 10, 38 or 48, wherein the distinct fluorescent groups comprise Redmond Red™, Yakima Yellow™, and the fluorescence-modifying group comprises an Eclipse™ non-fluorescent quencher, dabcyl, or other fluorescent-quenching molecule.

53. An improved assay according to claim 48, wherein the one or more target nucleic acid sequences is 2, each having a distinct SNP of interest.

54. An improved assay according to claim 53, wherein the one or more fourth primers is 2, each having a distinct fluorophore and unique sequence that incorporates into and amplifies each of the 2 target nucleic acid sequences.

55. An improved assay according to claim 48, wherein the one or more target nucleic acid sequences is 3, each having a distinct SNP of interest.

56. An improved assay according to claim 55, wherein the one or more fourth primers is 3, each having a distinct fluorophore and unique sequence that incorporates into and amplifies each of the 3 target nucleic acid sequences.

57. An improved assay according to claim 48, wherein the one or more target nucleic acid sequences is 4, each having a distinct SNP of interest.

58. An improved assay according to claim 57, wherein the one or more fourth primers is 4, each having a distinct fluorophore and unique sequence that incorporates into and amplifies each of the 4 target nucleic acid sequences.

59. A nanoliter sampling array comprising:
a) a first platen having at least one hydrophobic surface and having a high-density microfluidic array of hydrophilic through-holes;
wherein each first platen through-hole contains at least
i) one or more first primers, each having at least a portion substantially complementary to a first segment of one or more target nucleic acid sequences; and
ii) a second primer having at least a portion substantially complementary to a second segment of the one or more target nucleic acid sequences, the first and second primers being ligatable upon binding to the one or more target nucleic acid sequences.

60. A nanoliter sampling array according to claim 59, further comprising:
a second platen having at least one hydrophobic surface and having a high-density microfluidic array of hydrophilic second platen through-holes;
wherein the first and second platen are fixedly coupled such that the through-holes of each are aligned.

61. A nanoliter sampling array according to claim 59, wherein at least one pair of aligned through-holes contains at least first reagents for a first assay process and second reagents for a second assay process.

62. An array according to claim 61, wherein one of the assay processes is PCR amplification.

63. An array according to claim 61, wherein one of the assay processes is detection of one or more amplified target nucleic acid sequences, each having a SNP.

64. An array according to claim 63, wherein detection of one or more amplified target nucleic acid sequences comprises using one or more probes specific for hybridizing to a region of each of the one or more target sequences, each probe containing a distinct fluorescent group and a fluorescence-modifying group, wherein upon extension of the one or more probes into one or more amplified target nucleic acid sequences, each of the distinct fluorescence-modifying groups is excised and the distinct fluorescent group fluoresces.

65. An array according to claim 63, wherein detection of one or more amplified target nucleic acid sequences comprises using one or more probes specific for hybridizing to a region of each of the one or more target sequences, each probe containing a distinct fluorescent group and a fluorescence-modifying group, wherein the fluorescent group is quenched before incorporation into double-strand product and is dequenched after incorporation into double-stranded product.

66. An array according to claim 65, wherein the fluorescent group is quenched by secondary structure before incorporation into double-stranded product, such that before incorporation, a sequence in the probe binds to a complementary sequence in the probe containing the fluorescent group, quenching the fluorescent group.

67. A nanoliter sampling array according to any of claims 59-66, wherein the primers are affixed on, within or under a coating of the sample through-holes by drying, the coating comprising a biocompatible material.

68. A method of identifying one or more SNPs in one or more target nucleic acid sequences, the method comprising:
providing a first sample platen having a high-density microfluidic array of through-holes, each sample platen through-hole containing at least one or more first primers, each first primer having at least a portion substantially complementary to a first segment of the one or more target nucleic acid sequences, a second primer having at least a portion substantially complementary to a second segment of the target sequences, the 5′-end of the second primer ligatable to the 3′-end of the first primer after binding to the one or more target nucleic acid sequences, and a third primer that is substantially complementary to a random sequence segment of the second primer;
introducing a sample containing one or more target sequences of nucleic acid, each having a SNP of interest, to the sample platen through-holes in the array;
introducing reagents to the sample platen through-holes in the array, the reagents including a reagent for effecting amplification, a reagent for effecting ligation, and at least four different nucleotide bases;
effecting ligation of the first and second primers to produce a ligated product;
effecting amplification of the ligated product and one or more target sequences; and
detecting one or more amplified target sequences.

69. A method of identifying a SNP in a target sequence of nucleic acid according to claim 68, wherein effecting ligation and effecting amplification comprises addition of a ligase and a polymerase followed by subjecting the array to controlled-temperature conditions.

70. A method of identifying one or more SNPs according to claim 68, further comprising, before introducing reagents to the sample platen through-holes in the array:
introducing a sample containing one or more probes specific for hybridizing to a region of one or more target nucleic acid sequences and amplifying the one or more target sequences, wherein the one or more probes each contain a distinct fluorescent group and a fluorescence-modifying group.

71. A method of identifying one or more SNPs according to claim 70, wherein upon extension of the one or more probes into one or more amplified target nucleic acid sequences, each of the distinct fluorescence-modifying groups is excised and the distinct fluorescent group fluoresces.

72. An method of identifying one or more SNPs according to claim 70, wherein the fluorescent group is quenched before incorporation into double-strand product and is dequenched after incorporation into double-stranded product.

73. An method for identifying one or more SNPs according to claim 72, wherein the fluorescent group is quenched by secondary structure before incorporation into double-stranded product, such that before incorporation, a sequence in the probe binds to a complementary sequence in the probe containing the fluorescent group, quenching the fluorescent group.

74. A method according to claim 71, wherein identifying one or more SNPs in one or more target nucleic acid sequences comprises monitoring differential fluorescence of the one or more distinct fluorescent groups incorporated into the one or more amplified target nucleic acid sequences.

75. A method of identifying one or more SNPs in one or more target sequences of nucleic acid according to claim 68, wherein the polymerase lacks 5′ to 3′ exonuclease activity.

76. A method of identifying one or more SNPs in a target sequence of nucleic acid according to claim 68, further comprising using a polymerase that lacks 3′ to 5′ exonuclease activity.

77. A method of identifying one or more SNPs in one or more target nucleic acid sequences according to claim 68, further comprising using a polymerase that lacks 5′ to 3′ exonuclease activity and 3′ to 5′ exonuclease activity.

78. A method according to claim 70, wherein the one or more target nucleic acid sequences is 2, each having a distinct SNP of interest.

79. A method according to claim 78, wherein the one or more hybridizable probes is 2, each having a distinct fluorophore and unique sequence that hybridizes to and amplifies each of the 2 target nucleic acid sequences.

80. A method according to claim 70, wherein the one or more target nucleic acid sequences is 3, each having a distinct SNP of interest.

81. A method according to claim 80, wherein the one or more hybridizable probes is 3, each having a distinct fluorophore and unique sequence that hybridizes to and amplifies each of the 3 target nucleic acid sequences.

82. An improved assay according to claim 70, wherein the one or more target nucleic acid sequences is 4, each having a distinct SNP of interest.

83. An improved assay according to claim 82, wherein the one or more hybridizable probes is 4, each having a distinct fluorophore and unique sequence that hybridizes to and amplifies each of the 4 target nucleic acid sequences.

84. A kit for use in identification of one or more amplified target nucleic acid sequences, the kit comprising:
a) a sample platen having one hydrophobic surface and having a high-density microfluidic array of hydrophilic through-holes;
wherein each sample platen through-hole contains at least
i) one or more first primers, each first primer having at least a portion substantially complementary to a first segment of one or more target nucleic acid sequences;
ii) a second primer having at least a portion substantially complementary to a second segment of the one or more target nucleic acid sequences, the 3′-end of the one or more first primers ligatable to the 5′-end of the second primer after binding to the one or more target nucleic acid sequences;
b) a reagent platen having a high-density microfluidic array of through-holes, each reagent platen through-hole containing at least
i) a third primer that is substantially complementary to a random sequence segment of the second primer;
ii) one or more probes specific for hybridizing to a region of one or more target nucleic acid sequences and amplifying the one or more target sequences, wherein the one or more probes each contain a distinct fluorescent group and a fluorescence-modifying group;
iii) four different nucleotide bases;
iv) a ligase; and
the reagent platen having a structural geometry that corresponds to the sample platen allowing delivery of reagent components and target nucleic acid sample to the primers in the sample platen.

85. A kit for use in identification of amplified target nucleic acid sequences according to claim 84, wherein a PCR-compatible buffer is also included.

86. A kit according to claim 84 or 85, wherein the primers are affixed on, within or under a coating of the sample through-holes by drying, the coating comprising a biocompatible material.
